(19) United States
(12) Patent Application Publication
(10) Pub. No.: US 2016/0017288 A1
(43) Pub. Date: Jan. 21, 2016

(54) METHOD FOR INDUCING THE FORMATION OF ISLET STRUCTURES AND IMPROVING BETA CELL FUNCTION

(71) Applicant: Eitan Moshe Akirav, Plainview, NY (US)

(72) Inventor: Eitan Moshe Akirav, Plainview, NY (US)

(21) Appl. No.: 14/721,993

(22) Filed: May 26, 2015

Related U.S. Application Data
(60) Provisional application No. 62/003,572, filed on May 28, 2014.

(51) Int. Cl. C12N 5/071

(52) U.S. Cl. CPC C12N 5/0677; C12N 2502/22; C12N 2506/22

(57) ABSTRACT

Insulin producing β cells are found in three dimensional (3D) structures, the Islet of Langerhans. The 3D structure is required for normal β cell function and survival. β cell pseudoislets (PIs) are useful for study of β cell physiology. Co-culturing of primary human islets and β cell lines together with islet-derived epithelial cells can improve β cell function and survival and maintain the cells' 3D structure, resulting a rapid and spontaneous formation of free-floating PIs. β cells in PIs were similar in size to native islets and showed increased percentage of pro-insulin-positive cells, increased insulin gene expression in response to glucose stimulation, improved glucose-stimulated insulin secretion, and reduced β cell death. Key ECM proteins, absent in monolayer β cells, are deposited by iECs in and round the PIs. iEC induced PIs are a useful tool for examining β-cell/iEC interactions and studying β-cell function in a native 3D configuration.

### BACKGROUND

1. Field of the Invention

The present invention relates to the field of mammalian cell culture, and more particularly to advancements to in vitro beta islet cell culture.

2. Related Art

The islets of Langerhans are 3-dimensional (3D) structures that contain insulin producing β cells. In vivo, islets contain key components of the extracellular matrix (ECM) which can be found in the islet capsule and around the islet microvessels. The ECM is made of defined proteins that include collagens and laminins (1). In vivo, islet endothelial cells (ECs) are a major source for ECM proteins, and previous studies demonstrated an active role for ECs in mediating β cell function, by promoting β-cell differentiation and increasing insulin production (2, 3). Following isolation, primary islets lose both ECs and ECM. This leads to impaired islet function in vitro (4-6) and results in apoptosis (7, 11). Previous reports show that culturing of human islets (hIslets) on different ECM proteins enhances β cell function in vitro (8). Over time, however, cultured hIslets lose their 3D structure due to islet adhesion and flattening, resulting in a loss of islet phenotype (7, 8) and reduced insulin production (9).

Disruption of the islet structure impairs β-cell function by inducing β-cell dedifferentiation and reducing β-cell survival (4-6). The formation of 3-D β-cell aggregates, or pseudoislets (PIs), are used for the study of β cell biology as they improves β-cell function by increasing insulin production and improving glucose-stimulated insulin secretion (GSIS) (11-17). These effects are mediated in part by the formation of a 3-D configuration shown to enhance β cell-cell contact (12, 16), increase calcium signaling (18), and preserve extracellular matrix proteins (19). Despite their usefulness, PI generation requires extensive cell manipulation and may take several weeks to form (7-14 d). Current methods for induction of PIs include the use of mechanical manipulations, such as stirred cell suspension cultures (15), culturing of β-cells on gelatin coated plates (11), and hanging drop cell cultures (20).

The islet endothelium plays a critical role in β-cell function and survival (5). Changes in islet endothelial cell (iEC) density and activation are associated with altered β-cell function under physiological and pathological conditions. The control of β-function and mass is partially mediated by iEC ability to produce pro-β cell factors (21) and support the islet structure via the depositions of ECM proteins, such as collagen IV (col-IV) and laminin (22-23). In isolated human islets the addition of ECM proteins delays β-cell dedifferentiation while maintaining insulin expression (8).

### SUMMARY

β-cells grown as monolayers show reduced insulin production and glucose responsiveness due to loss of 3D configuration and key extracellular matrix components (ECM). β cell lines, such as βTC3 and MIN6, show improved insulin production and insulin release when grown in the presence of ECM proteins and can form 3D structures, also known as pseudoislets (PIs). The islet endothelium (EC) is important in the production of ECM and is important for normal β cell function.

The present technology uses islet-derived ECs to maintain islet structure and function in vitro. Co-culturing of primary human islets and β cell lines together with islet-derived ECs can improve β cell function and survival and maintain the cells' 3D structure. In β cell lines, islet-derived ECs are capable of supporting the formation of free-floating islet-like structures, while improving insulin production in the β cells. More importantly, islet-derived ECs also increase ECM deposition, without inducing islet attachment in primary human islets.

The present technology provides a method for utilizing islet-derived ECs for maintaining islet structure and preserving β cell function in long term free-floating primary human-islet cultures.

The present technology also provides a system and method for co-culturing insulin producing β-cells and islet derived ECs to induce the formation of free-floating pseudoislets (PI) and improve β cell function, as well as the pseudoislets so formed.

One aspect of the technology provides a method for forming free-floating primary islet cultures comprising co-culturing islet β cells with islet-derived epithelial cells.

The islet cell cultures may be used in vitro to produce various hormones. The islets might also be transplanted in vivo as a therapy for islet dysfunction, e.g., diabetes. In some cases, an autotransplant may be employed, where β cells generated from stem cells from the afflicted individuals are co-cultured with primary islet derived ECs ex-vivo to expand the stock establish a supply of pseudoislets for re-implantation into the patient, e.g., on the pancreas, thus avoiding need for immunosuppressive drugs. The pseudoislets may also be used in research and therapies which comprise artificial organs, which may be extracorporeal or implanted in a patient.

A straightforward and rapid method for inducing free-floating PIs by co-culturing iEC and β-cell insulinoma lines is provided. Newly formed PIs are positive for ECM proteins produced by iECs and show improved insulin production, insulin sensing and glucose stimulated insulin secretion when compared with monolayer cells. iEC-induced PIs may serve as a useful tool for examining β-cell/iEC interactions and studying β-cell function in a native 3D configuration.

### BRIEF DESCRIPTION OF THE DRAWINGS

FIGS. 1A, 1B, 1C and 1D show co-culturing of MS-1 and βTC3 cells results in the formation of free floating insulin positive islet like structures (aIslet), in which FIG. 1A shows light and phase contrast microscopy showing the formation of islet like structures in a monolayer stage, at 24 hrs., 48 hrs., 72 hrs., Day 8, and as a pseudoislet; FIG. 1B shows IF staining of structures. i-DAPI, ii-Insulin, III-merge. FIG. 1C shows a confocal image, representing a 3D Z-stack reconstruction of insulin positive cells, of βTC3 monolayer and aIslet, in which Blue is DAPI, and Red is Insulin; and FIG. 1D shows real Time PCR analysis of insulin 1 expression under increasing glucose concentrations.

FIGS. 2A, 2B and 2C show cultured Islet derived EC line (MS-1) are positive for collage IV and laminin, in which FIG. 2A

shows RT-PCR for laminin β1 and collagen IV in MS-1, whole murine islet preps, and murine insulinoma line βTC3 (Laminin α1 and α2 were not detected, data not shown); FIG. 2B shows IF staining of MS-1 cells (i-DAPI, ii-Collagen IV, III-BS-1, IV-Laminin, and V-merge); FIG. 2C shows IF staining of 7d PIs from cocultures of MS-1 and βTC3 cells (i-DAPI, ii-Insulin, iii-Col-IV, iv-Laminin, v-Merge).

FIGS. 3A, 3B and 3C show pseudoislets having improved insulin staining and increased cell prolifeation, by FACS analysis of βTC3 monolayers (dotted line) and pseudoislets (solid line), in which FIG. 3A shows FACS staining for pro-insulin expression in monolayers (dotted line) and PIs (solid line), with a grey histogram-isotype control; FIG. 3B shows propidium iodide staining; and FIG. 3C shows Ki67 staining.

FIGS. 4A and 4B show increased ECM deposition and insulin staining in vitro by co-culturing of islet-derived EC line MS-1 and primary human islets, in which FIG. 4A shows 8 day cultures of hIslet (White arrows point to high Col IV and laminin deposition in the hIslets); FIG. 4B shows pixel intensity quantization of insulin positive cells in hIslets cultured alone or in the presence of MS-1 cells.

FIGS. 5A, 5B and 5C show in vitro expanded primary ECs from human islets cultured on collagen coated plates express collagen IV and induction of the formation of islet like structures, in which FIG. 5A shows a fresh free floating islet; FIG. 5B shows primary human islets which were cultured on collagen coated plates; and FIG. 5C shows IF staining of primary islet derived hECs (i-DAPI, ii-Collage IV, iii-BS-1).

FIGS. 6A-6B shows that MS induces a spontaneous formation of free-floating insulin-positive PIs. FIG. 6A. IF staining of βTC3 monolayers. Blue-DAPI, Red-Insulin and merge. FIG. 6B shows a 3-D reconstruction of Z-stack confocal images of a representative PI (Blue-DAPI, Red-Insulin, and merged).

FIGS. 7A-7B show baseline and glucose stimulated insulin expression and secretion are enhanced in MS1-induced PIs. FIG. 7A shows quantitative RT-PCR analysis of insulin expression in βTC3 monolayers (closed bars) or PIs (opened bars), with data representing an average of 3 independent experiments. (*p<0.025). FIG. 7B shows an insulin ELISA analysis of supernatant from βTC3 monolayers or PIs, in which the experiment represents three independent repeats (N=3 per group, 2-way ANOVA analysis, †p<0.001, *p<0.0001).

FIGS. 8A-8D show that Col-IV and laminin are detected in and around the PI. FIG. 8A shows IF staining of MS1 cells (Blue-DAPI, Green-CD31, Red-BS1 and merge; White/Yellow represents double positive cells). FIG. 8B shows IF staining of MS1 cells (Blue-DAPI, Green-Col-IV, White-Laminin, and merge). FIG. 8C shows a 3-D reconstruction of Z-stack imaging of an 8 day old PI. FIG. 8D shows non-consecutive Z-stack confocal images of PI (Blue-DAPI, Red-Insulin, Green-cleaved caspase-3).

FIGS. 9A-9B show evaluation of β cell loss in MS1-induced PIs. FIG. 9A shows FACS staining for 7-AAD viability dye of monolayers (dotted line) and PIs (solid line); Grey histogram unstained control; Black arrow show PI positive cells. FIG. 9B shows non-consecutive Z-stack confocal images of 14 day old PI (Blue-DAPI, Red-Insulin, Green-cleaved caspase-3; White arrow point to caspase-3 positive cells).

### DETAILED DESCRIPTION OF THE PREFERRED EMBODIMENTS

### Example 1

### Generation of Pseudoislets (PIs) Using Islet Derived Murine Endothelial Cells and Insulinoma Cell Lines

The following procedure describes the formation of PIs using islet derived ECs (MS-1 cells).

This procedure allows for the formation of free floating islets which contain ECM components and show improved insulin production as described Spelios et al. (36 and 37).

βTC3 is a murine-derived insulinoma cell line that does not produce ECM proteins. To evaluate the effects of ECM on βTC3 cells, we established a co-culture system of βTC3 cells and the islet-derived EC line, MS-1. RT-PCR and immunofluorescence (IF) staining showed expression of both laminin and collagen IV in MS-1 cells. Mixing of MS-1 and βTC3 cells resulted in the formation of free-floating islet-like structures as early as 48 h following co-culture, while βTC3 cultured alone remained attached to the surface as a monolayer. Confocal microscopy showed the deposition of laminin and collagen IV on the surface of newly formed pseudo islets. FACS analysis showed increased percentage and mean fluorescence intensity (MFI) staining of proinsulin and Ki67 in pseudo islets when compared with βTC3 cultured alone. The formation of 3-D βTC-3 structures following co-culture enhanced insulin gene expression in response to glucose stimulation when compared with monolayers. The findings demonstrate the ability of islet-derived ECs to deposit key ECM proteins and induce the formation of 3-D free-floating islet-like structure in βTC3 cells. These newly formed structures increased insulin production, improved glucose responsiveness, and increased cell proliferation, thus, providing a new method for examining β cell and EC interactions and enhance β cell function in vitro.

Generation of Peudoislets (PIs) from βTC3 Insulinoma Cells

MS1 murine iECs (29) were obtained from the American Type Culture Collection (Manassas, Va.) (MS-1 islet derived endothelial cells (ATCC Catalog number CRL-2279). βTC3 murine insulinoma cells at passage number 40-55 were previously described (30) and were a kind gift from Dr. Kevan Herold (Yale University, New Haven, Conn.).

βTC3 cells were cultured in Dulbecco's Modified Eagle's Medium (DMEM) containing 25 mM glucose and supplemented with 4.4 mM sodium bicarbonate, 15 mM HEPES, 1% penicillin/streptomycin/neomycin mixture, 15% heat-inactivated horse serum, 2.5% FetalClone II, and 1% Eagle's Minimum Essential Medium with nonessential amino acids. MS1 cells were also cultured under hyperglycemic conditions in DMEM modified with 5% heat-inactivated fetal bovine serum (FBS), 1% antibiotic mixture, and 0.25 μg/mL amphotericin B. All cell cultures were kept at 37° C. in a 5% CO2 in air humidified atmosphere.

For PI formation, β-cell/iEC co-cultures were prepared by seeding low passage βTC3 (2×10⁵ cells/well) and MS1 (6×10⁵ cells/well) in a 6-well tissue culture plate. The co-cultures were maintained at 37° C. for 7 d in DMEM supplemented with 25 mM glucose, 10% heat-inactivated FBS, 1% antibiotic mixture, 0.25 μg/mL amphotericin B, and 1 mM sodium pyruvate. Beta-cell monolayers were propagated concomitantly under the same growth conditions as the βTC3/MS1 co-cultures.

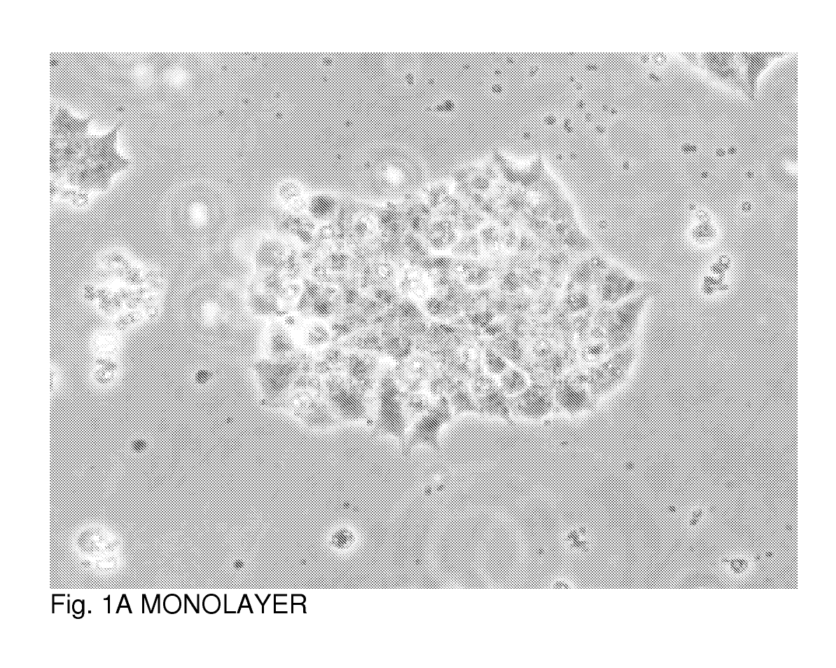

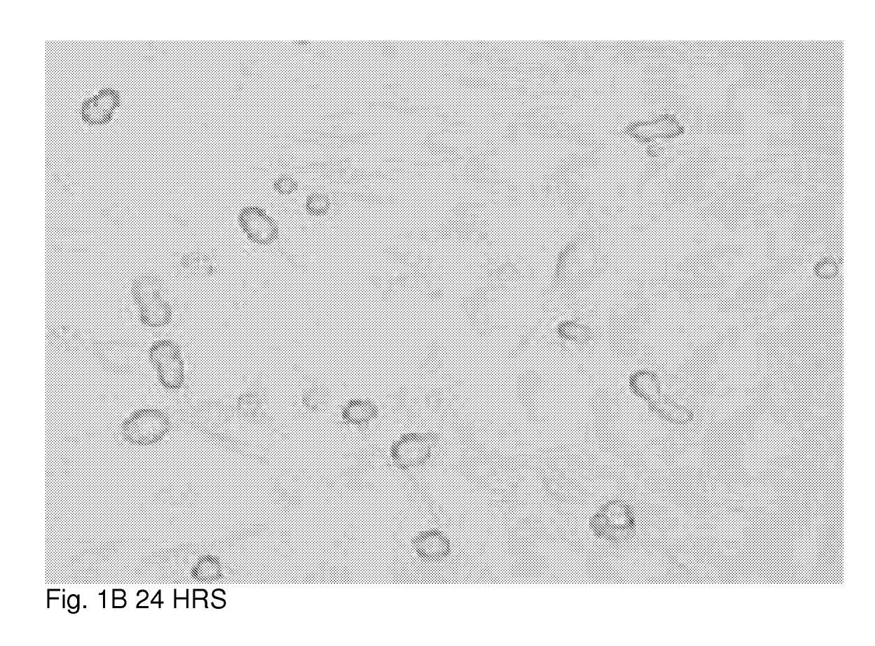

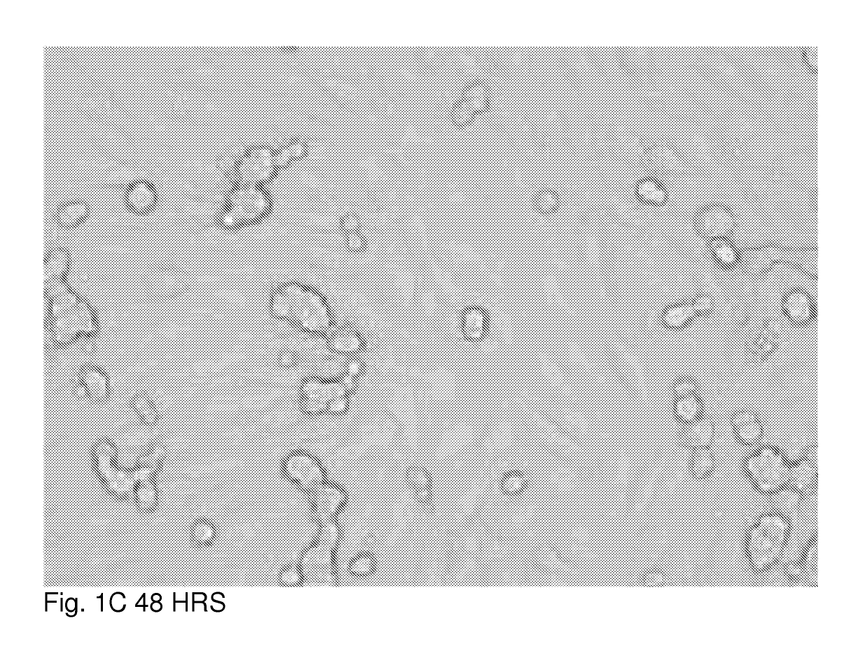

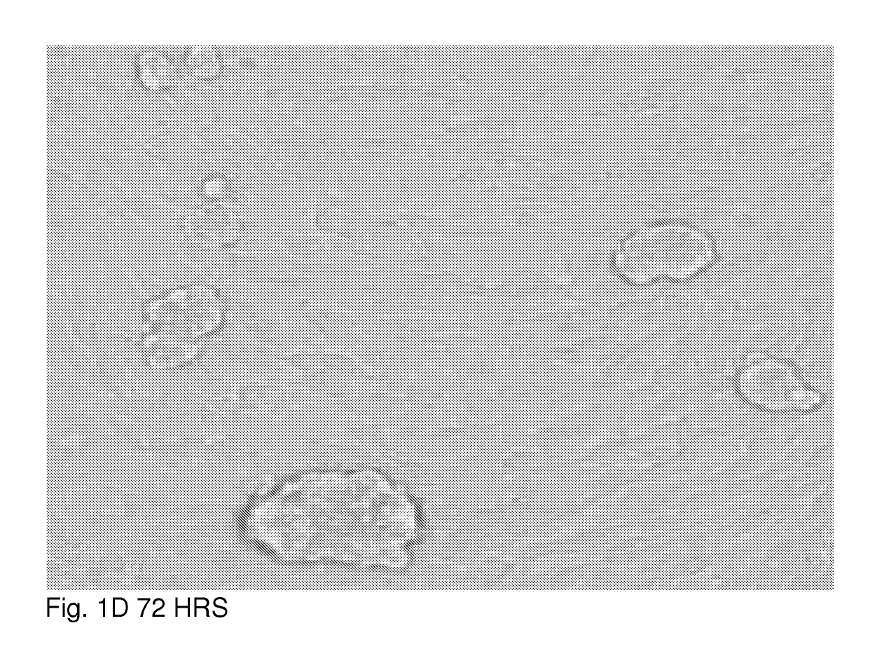

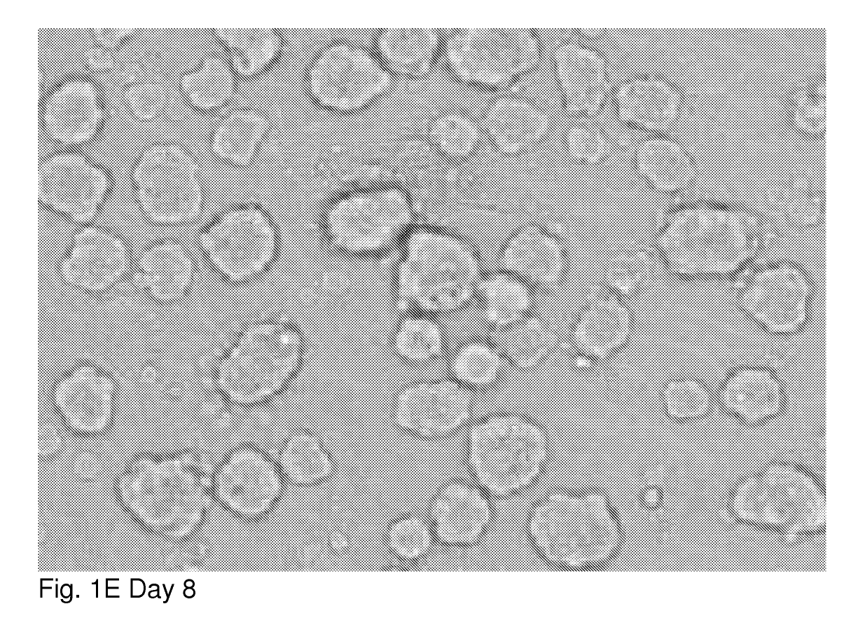

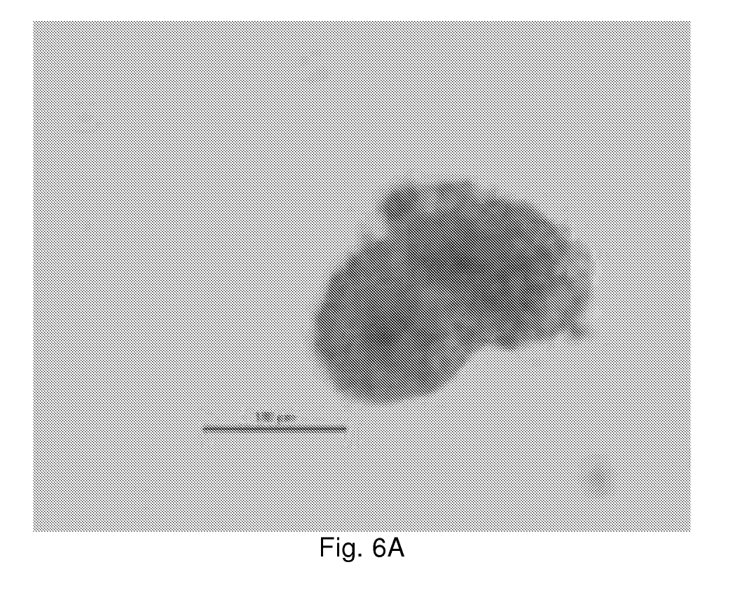

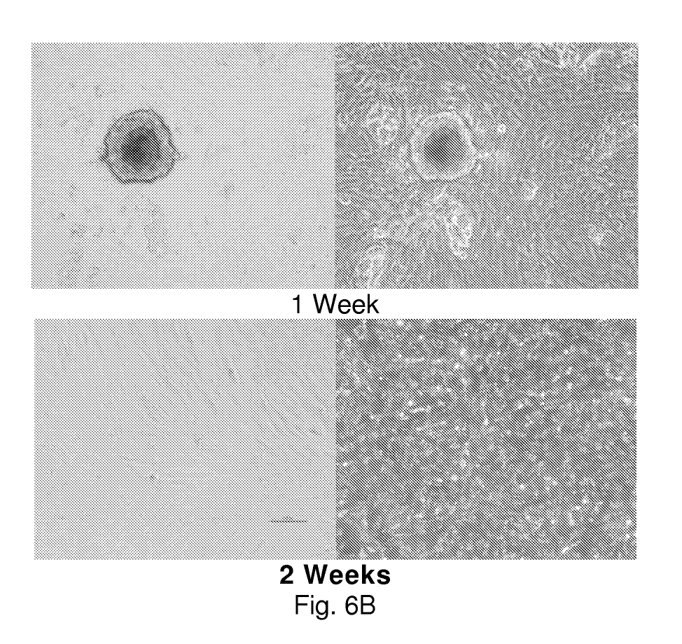

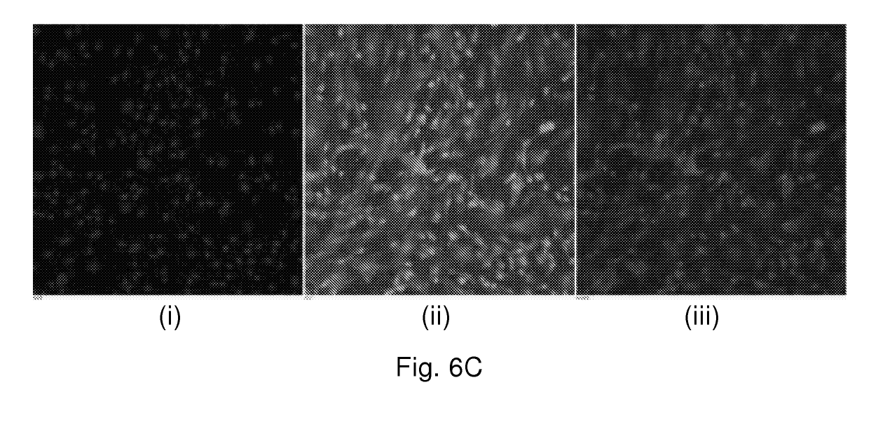

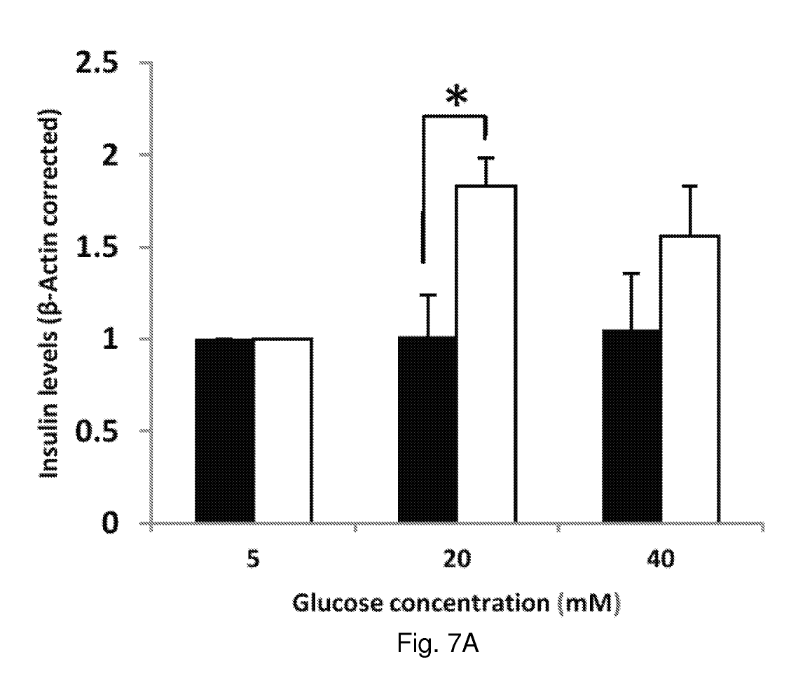

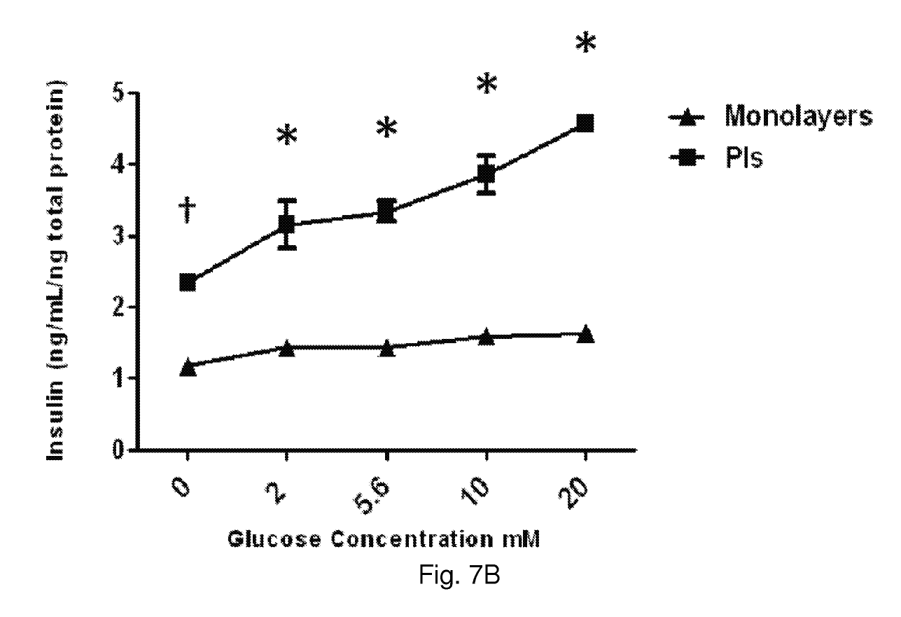

iEC's thus induce a spontaneous formation of free-floating PIs. The islet endothelium plays an important role in the formation of the islet structure and β-cell function (24). βTC3 cultured in the presence of MS1 cells formed cell clusters as soon as 24 h, and PI formation is detected as early as 48 hours after culture setup. The cell clusters spontaneously detached by 72 h forming free-floating PIs (FIG. 1A). PI density was increased by day 8 in culture (FIG. 1A), showing a spheroid structure ranging from 50 to 300 μm in size, similar to that of native murine islets (25). In contrast, βTC3 cells cultured alone remained adherent and failed to form PIs (FIG. 1A). Monolayer βTC3 cells stained positive for insulin (FIG. 6A). Similarly, PIs showed strong insulin staining, albeit in a 3-D structure (FIG. 6B). Interestingly, MS1 endothelial cells were not detected in or around the PIs as indicated by the absence of CD31 and BS1 staining (data not shown). Taken together, these results demonstrate a novel method for a rapid and spontaneous formation of free-floating PIs by co-culturing iECs and β-cells in vitro.

Monolayers and PIs were stained as follows. Monolayer MS1 and βTC3 cells were grown on glass bottom Petri dishes coated with poly-D-Lysin. Prior to staining, cells were washed in PBS and fixed in 2% PFA. For PIs, free floating PI were washed and fixed in 2% PFA in suspension. Following fixation, monolayer cells and PI were stained with or without primary antibodies to insulin (Invitrogen, Carlsbad, Calif.), col-IV (Abcam, Cambridge, Mass.), laminin (Abcam, Cambridge, Mass.), BS-1 (Sigma Aldrich, Saint Louis, Mich.), or cleaved caspase-3 (Cell Signaling Technology, Danvers, Mass.) along with 4′,6-diamidine-2-phenylindole dihydrochloride (DAPI). Secondary antibodies were used for detection of primary antibodies (Jackson Immunoresearch, West Grove, Pa.). Some specimens were stained using secondary antibodies only without primary antibody staining and used as for non-specific staining controls. Stained cells and PIs were analyzed using a Nikon Eclipse Ti confocal microscope (Nikon, Melville, N.Y.). Z-stack confocal imaging and reconstruction was used to evaluate PI structure and characterize βTC3 cell configuration inside the isle core.

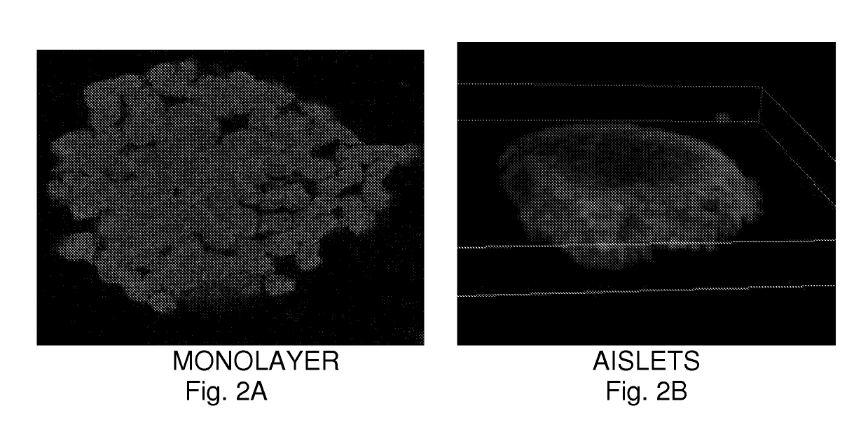

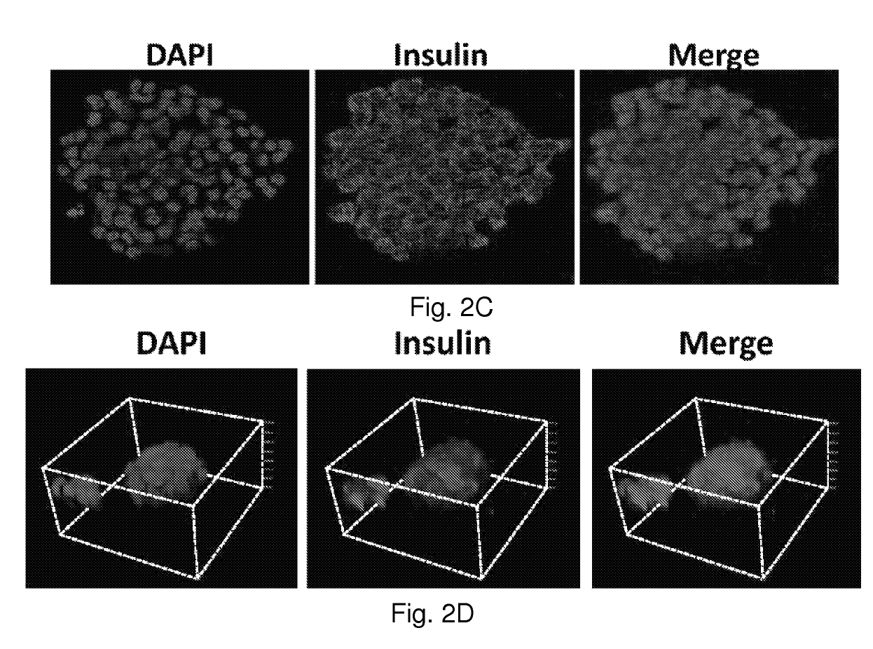

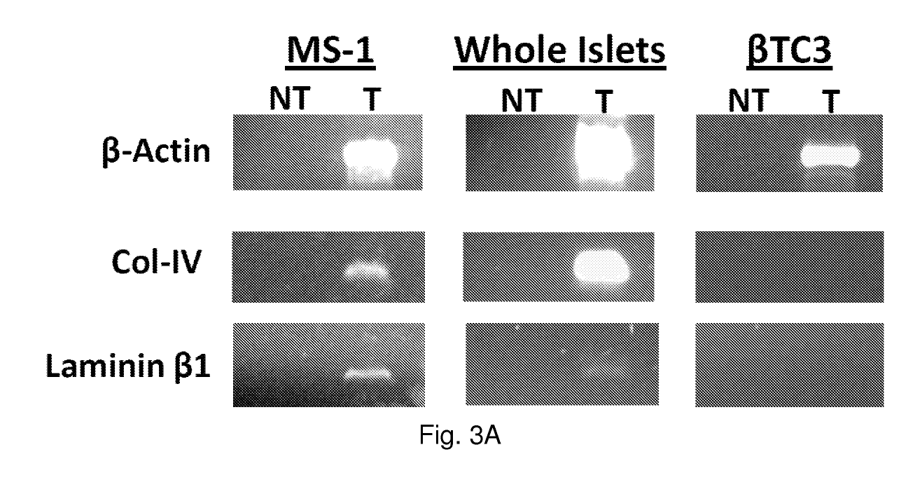

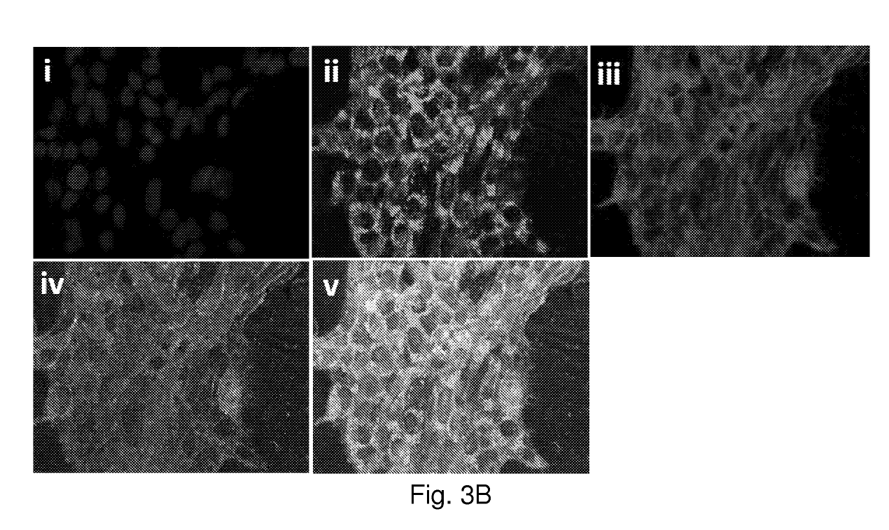

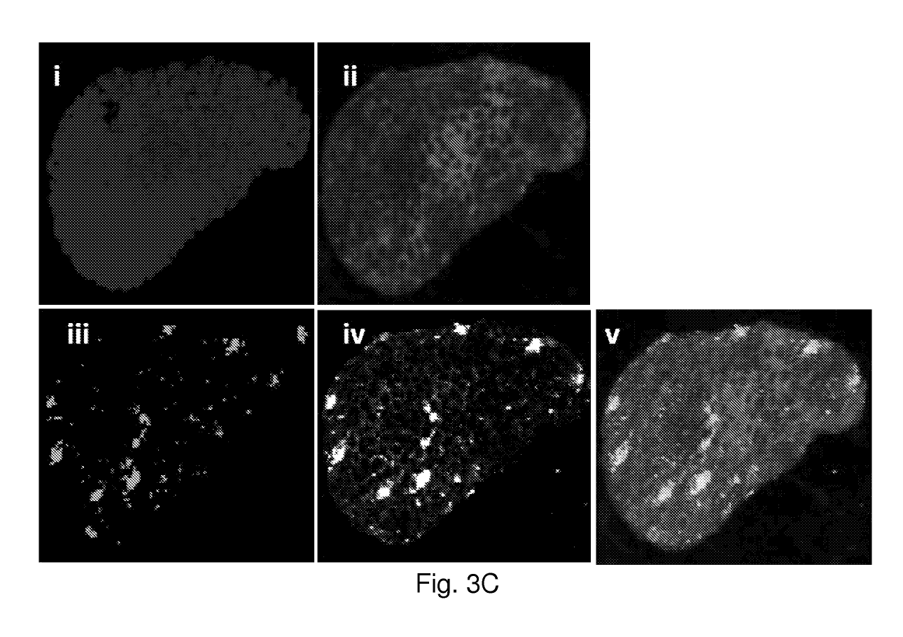

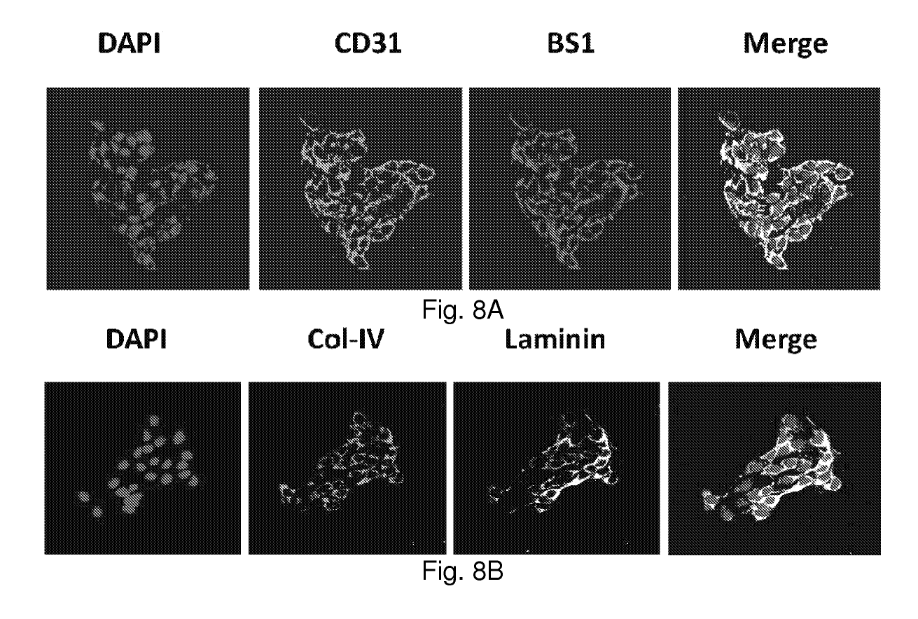

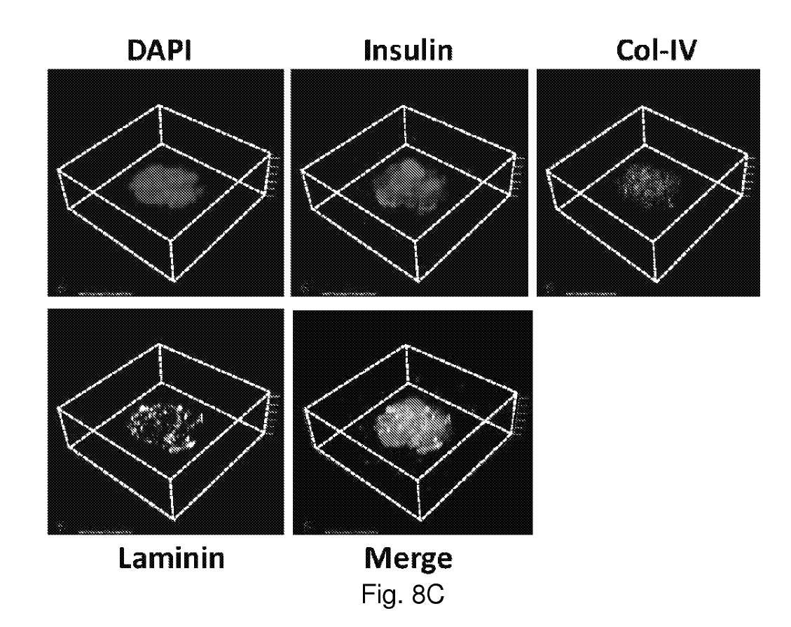

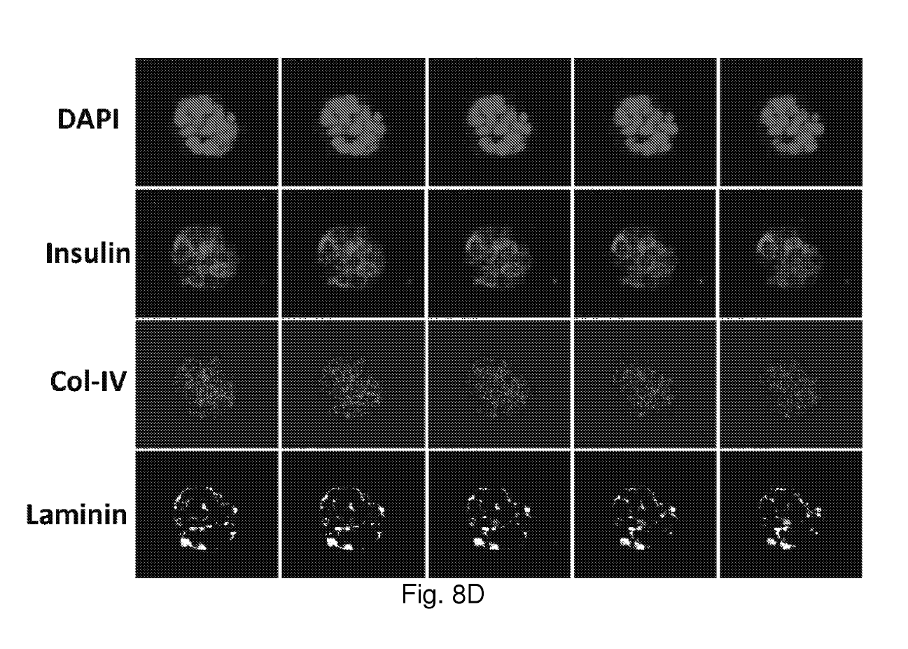

PIs are positive for laminin and col-IV and do not show increased β-cell death over time. ECM proteins, such as laminin and col-IV, are produced by iECs and are an integral part of the islet structure (22). iEC MS1 cells were tested to see if they express markers of native iECs by staining for CD31 (PECAM-1) and BS1 (lectin), as well as, laminin and col-IV. MS1 cells were highly positive for the endothelial cell markers, CD31 and BS1 (FIG. 3A). RT-PCR analysis detected the expression of laminin-β1 and col-IV mRNAs in MS1 cells and purified primary murine islets, but not in βTC3 cells (FIG. 2A). The expression of ECM proteins was further validated by IF staining showing strong collagen-IV and laminin expression in MS1 cells (FIG. 8B). The ability of MS1 cells to produce col-IV and laminin and the absence of these proteins in βTC3 cells cultured alone prompted an examination of whether laminin and col-IV are found in MS1-induced PIs. Z-stack confocal imaging showed clear staining of laminin and col-IV in and around the surface of the PIs (FIG. 8C). Nonconsecutive Z-stack images showed diffused col-IV staining while laminin staining exhibited a more punctuated pattern (FIG. 8D). No staining was observed in PI stained with secondary antibodies only (data not shown).

Determination of insulin positivity and cell death was done by fluorescence-activated cell sorting (FACS) analysis using a C6 flow cytometer (BD Biosciences, Ann Arbor, Mich.). For insulin, βTC3 monolayer and dispersed PI cells were permeabilized (Fix & Perm, Life Technologies, Grand Island, N.Y.) and stained with either biotin-conjugated monoclonal mouse anti-proinsulin antibody (Clone 253627, no reactivity with mature insulin, R&D Systems, Minneapolis, Minn.) or biotin-conjugated isotype control. Cy3-conjugated streptavidin (Jackson ImmunoResearch Laboratories, West Grove, Pa.) was used as to detect insulin positive cells. To determine βTC3 viability, none-permeablized cells were stained with 7-AAD (BD Biosciences, San Jose, Calif.).

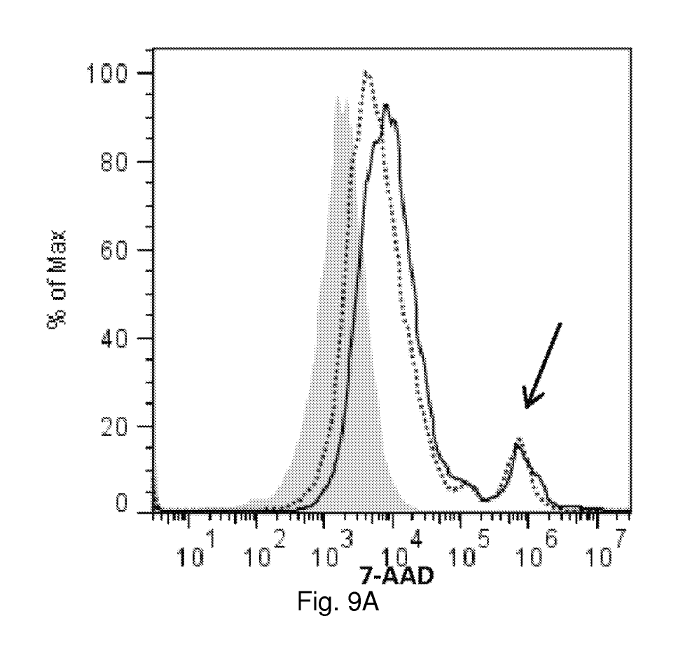

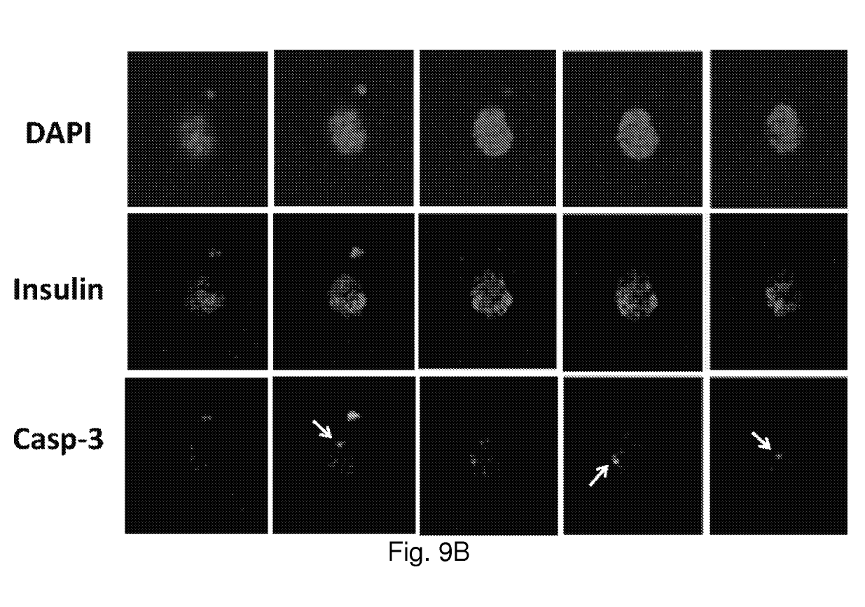

PI formation may lead to increased β-cell death over time (26). Such an increase is attributed to the development of a necrotic core at the center of the PIs. Therefore, β-cell death was examined to determine whether it was increased in PIs when compared with monolayer cells. FACS analysis using the viability dye 7-AAD showed similar levels of β-cell death in PIs and βTC3 monolayers (FIG. 9A). Confocal imaging of intact 14 day old PIs failed to reveal a necrotic core while showing strong and ubiquitous insulin staining throughout the PIs (FIG. 9B). Similarly, staining for cleaved caspase-3, a marker of cell apoptosis, detected only a small numbers of scattered apoptotic cells in the PIs; however these cells were not confined to the PI core (FIG. 9B). Taken together, these data suggest MS1-induced PIs contain newly deposited ECM proteins and do not display an increase in β-cell death while exhibiting an intact insulin-positive PI core.

In contrast to monolayer β-cells, PIs represent a more native culture condition for β-cells (27). Indeed, β cell cultured as PIs have improved function, partially attributed to enhanced cell-cell contact leading to increased glucose sensing, insulin production and insulin release (11-16). Despite their usefulness, the formation of PIs requires specialized culture conditions and extensive cell manipulation. A method for rapid and spontaneous induction of free-floating PIs is provided by co-culturing iECs and β cells. iEC-induced PIs have increased insulin synthesis and improved glucose responsiveness, maintain cell viability over time, and are positive for ECM proteins normally found in native islets. This method of PI formation provides a tool for the study of β-cell physiology in a 3-D conformation, while improving glucose sensing and β-cell viability. It offers a convenient way for examining the direct interactions between iECs and β-cells in vitro.

Current methods of PI formation require extensive cellular and mechanical manipulations of β-cells, thus limiting their use (11,15,20). Co-culturing of MS1 cells together with βTC3 cells resulted in a rapid and spontaneous formation of free-floating PIs, as early as 72 h in culture. This relatively short duration represents an improvement over previous methods requiring 7-14 days or longer for PI formation (11,15,20). The spontaneous detachment of iEC-induced PIs provides an additional advancement over previous methods, by eliminating the mechanical detachment of the PIs. PI detachment may be partially mediated by differences in the rate of cell division between iECs and β-cells. MS1 cells show a high turnover rate allowing the cells to reach a near 100% confluency by 24-48 h in culture. In contrast, βTC3 cells require 72-96 h before reaching full confluency. This difference in cell proliferation may limit the available area for βTC3 expansion, thus promoting the formation of β-cell clusters and subsequent detachment of free-floating PIs. Indeed, the addition of βTC3 cells to a fully confluent MS1 monolayer results in the formation of PIs similar to those formed with mixed cultures, albeit at a faster rate (48-72 h, data not shown).

Both iECs and the ECM is an integral part of the islet (28). The islet endothelium is a source of ECM in the islet (29). In vitro culturing of β-cells on ECM-coated surfaces can increase β-cell function and improve insulin production and glucose responsiveness (8). MS1, but not βTC3 cells, can produce laminin and col-IV in vitro. Both proteins were found in and around the PIs, suggesting the continuous deposition of ECM proteins during PI formation. The punctuated pattern of laminin deposition suggests that the process of ECM deposition differs from that of a native islet and is most likely mediated by direct cell-cell contact between βTC3 and MS1 cells during PI formation. The accumulation of ECM proteins may further improve β-cell contact and support cell adhesion, both of which can improve β-cell function over time (30). Interestingly, PIs did not include MS1 cells. The absence of MS1 cells in the PIs may relate to the relatively fast detachment of the PIs from the surface of the culture plate. Alternatively, the ability of MS1 cells to produce VEGF in an autocrine manner (data not shown), may preserve MS1-MS1 contact, thereby preventing the attachment of MS1 cells to the PI surface. In summary, PIs induced by iECs show the deposition of native ECM proteins in and around the PIs. This phenomenon offers a method for the inclusion of key ECM proteins involved in β-cell function and differentiation.

PI formation improves β-cell function overtime (11-16). MS1-derived PIs include an increased proportion of insulin-positive cells when compared with βTC3 monolayers. This increase in insulin-positive cells was associated with enhanced insulin gene transcription and improved insulin secretion in response to escalating glucose concentrations. The augmented insulin production and glucose sensing in PIs have been previously reported. Kitsou-Mylona et al. showed that improved glucose sensing in PIs correlated with improved extracellular calcium sensing via the extracellular calcium-sensing receptors Car2 and Car4 (31). Disruption of the Car pathway attenuated insulin secretion in response to glucose. Cell adhesion receptors such as E-cadherin and connexin are also involved in calcium oscillation, as the disruption of gap junctions can negatively affect insulin secretion of PIs (18,32). This enhanced cell-cell contact in PIs and increased calcium signaling may provide a mechanism whereby insulin expression and release is increased in PIs. PI formation is often correlated with increased β-cell death and reduced β-cell proliferation. Luther et al. showed an overall increase in β-cell death which was manifested by the formation of a necrotic core at the center of the PIs (26). Reers et al showed that MIN6 PIs exhibit an overall reduction in β-cell proliferation (33). These changes in β cell viability and proliferation represent a limitation for the use of PI in the study of β cell physiology. In the model described herein, β-cell death was unaffected in PIs as we did not detect the formation of a necrotic core over time. These differences may be explained by the presence of a pro-β-cell factor(s) produced by MS1 cells (24). The identification of novel factors produced by iECs and the ability of these cells to induce the formation of PIs may offer a new way for induction of islet like structure and improved β cell function in models involving primary β cell formation, such as stem cell derived β cells. A method for a rapid and spontaneous induction of PIs using co-cultures of iECs and β insulinoma cells is therefore provided. iEC-derived PIs have integrated ECM proteins and show improved insulin production, enhanced glucose responsiveness and improved glucose-stimulated insulin secretion. iEC-induced PIs may serve as a useful tool for examining β-cell/iEC interactions and studying β-cell function in a native 3D configuration.

Generation of PIs from MIN6 Insulinoma Cells

400,000 MIN6 cells are mixed together with 600,000 MS-1 and DMEM, high glucose, 10% FCS, P/S/N. The cells are plated in a single well of a 6 well plate, and the plate is then placed in a 37° C. 5% CO₂ incubator.

PI formation is detected as early as 96 hours after culture setup. Free floating PIs are harvested and moved to a Petri dish for additional analysis.

FIGS. 1A, 1B, 1C and 1D show co-culturing of MS-1 and βTC3 cells results in the formation of free floating insulin positive islet like structures (aIslet). FIG. 1A shows light and phase contrast microscopy showing the formation of islet like structures over a period of 48 hrs. Free floating islet like cells can be maintained for several weeks in culture. FIG. 1B shows IF staining of structures. i-DAPI, ii-Insulin, III-merge. FIG. 1C shows a confocal image of βTC3 monolayer and aIslet. Blue-DAPI, Red-Insulin. aIslet is a 3-D Z-stack reconstruction of insulin positive cells. FIG. 1D shows Real Time PCR analysis of insulin 1 expression under increasing glucose concentrations.

FIGS. 2A, 2B, and 2C show cultured Islet derived EC line (MS-1) are positive for collage IV and laminin. FIG. 2A shows results of RT-PCR for laminin β1 and collagen IV in MS-1, whole murine islet preps, and murine insulinoma line βTC3. Laminin α1 and α2 were not detected (data not shown). FIG. 2B shows IF staining of MS-1 cells. i-DAPI, ii-Collagen IV, III-BS-1, IV-Laminin, and V-merge. FIG. 2C shows IF staining of 7d PIs from cocultures of MS-1 and βTC3 cells. i-DAPI, ii-Insulin, iii-Col-IV, iv-Laminin, v-Merge.

FIGS. 3A, 3B and 3C show improved insulin staining and increased cell proliferation in peudoislets (PI). The graphs show FACS analysis of βTC3 monolayers (dotted line) and pseudoislets (solid line). FIG. 3A shows intracellular proinsulin staining FIG. 3B shows propidium iodide staining FIG. 3C shows Ki67 staining.

### Example 2

### Maintaining Insulin Production and ECM Proteins by Co-Cultures of Islet Derived Murine Endothelial Cells and Primary Human Islets

The following procedure describes the effects of MS-1 cells on insulin production and ECM deposition in primary human islets.

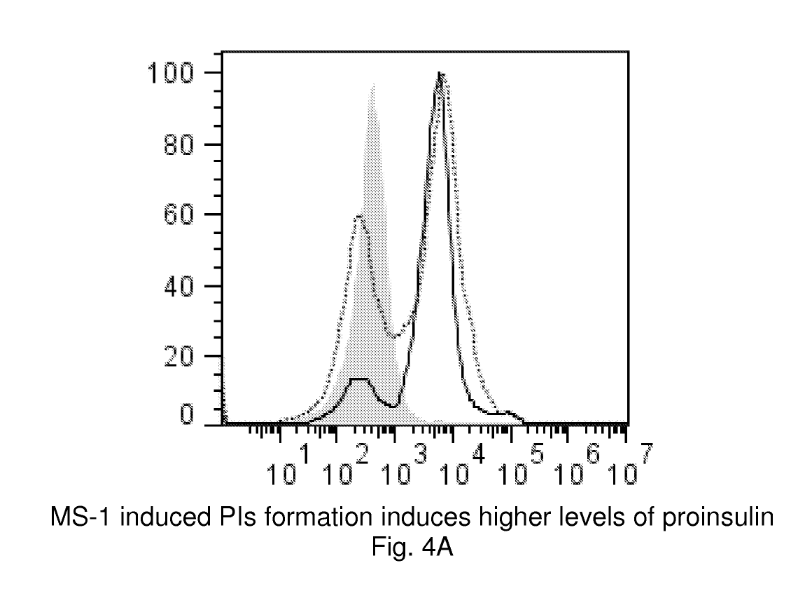

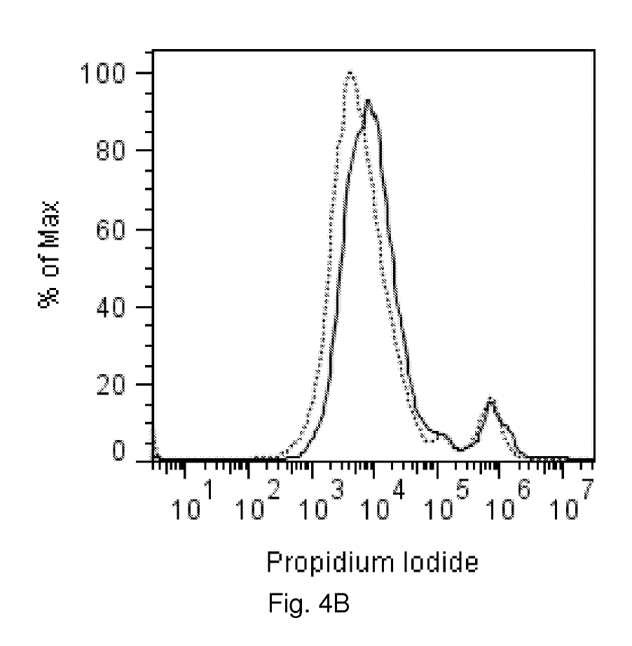

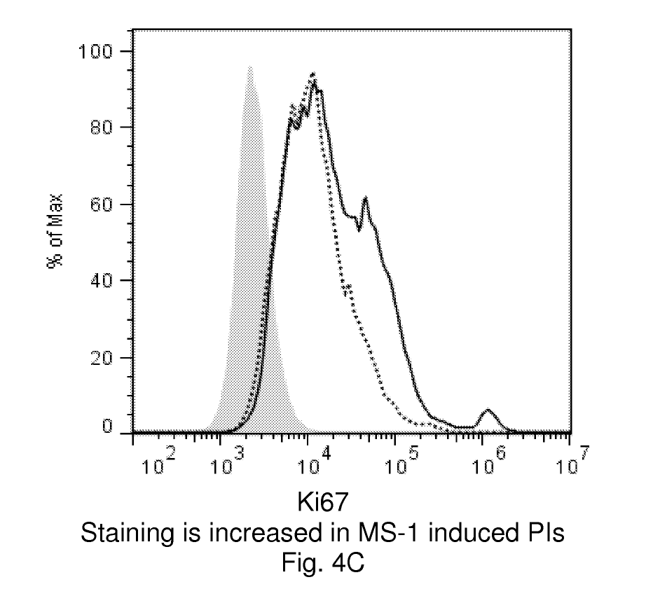

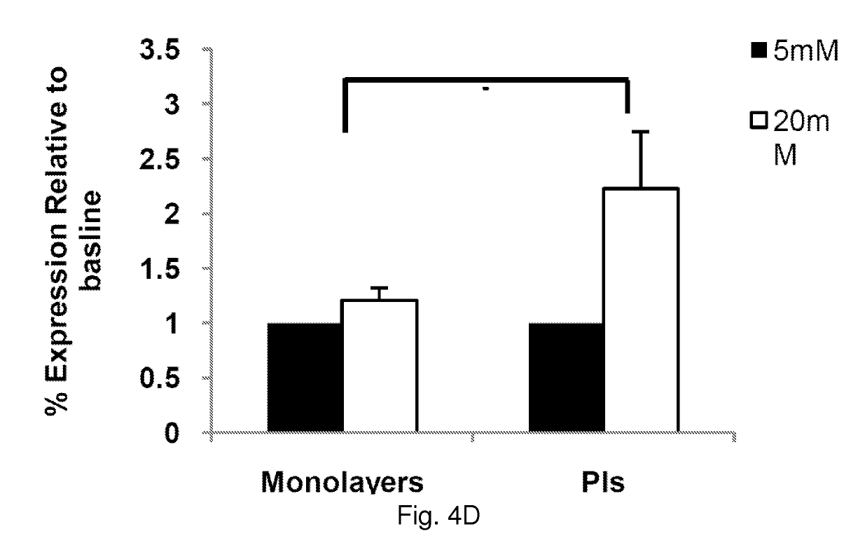

Procedures (See, FIGS. 4A-4B)

MS-1 cells are grown to ˜100% confluency in T-12.5 tissues culture treated flask. Primary hIslets (Primary human islets—1000 IEQ) are added to the flask containing the MS-1 cells. The flask is placed on an orbital shaker in a 37° C. 5% CO₂ incubator running at 70 rpm. Media (DMEM, 5 mM glucose, 10% FCS, P/S/N) is replaced every 4 days.

FIGS. 4A and 4B show co-culturing of MS-1 and primary human islets increased ECM deposition and insulin staining in vitro. Human primary islets were cultured alone or in the presence of murine islet derived ECs (MS-1). FIG. 4A shows 8 day cultures of hIslet. White arrows point to high Col IV and laminin deposition in the hIslets. FIG. 4B shows pixel intensity quantization of insulin positive cells in hIslets cultured alone or in the presence of MS-1 cells. Analysis was done using ImageJ histomorphic analysis software.

### Example 3

### Generating Human Islet Derived ECs to Increase the Viability and Function of Primary Human Islets

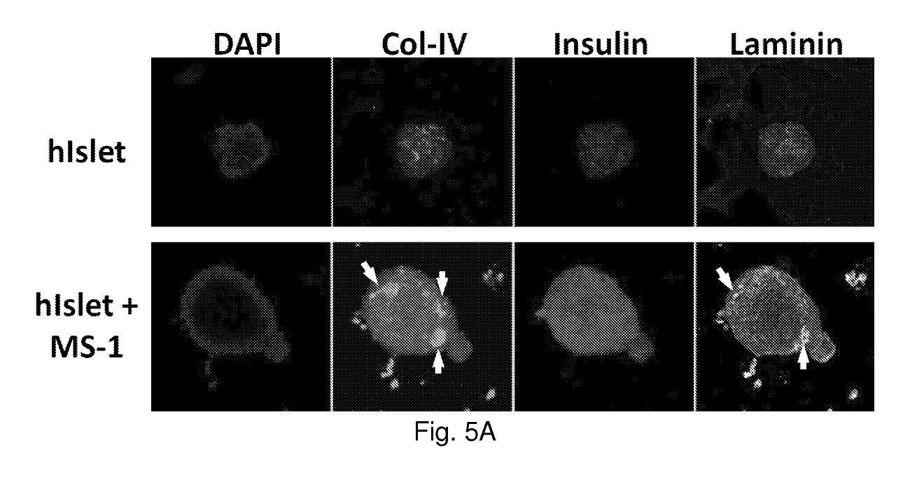

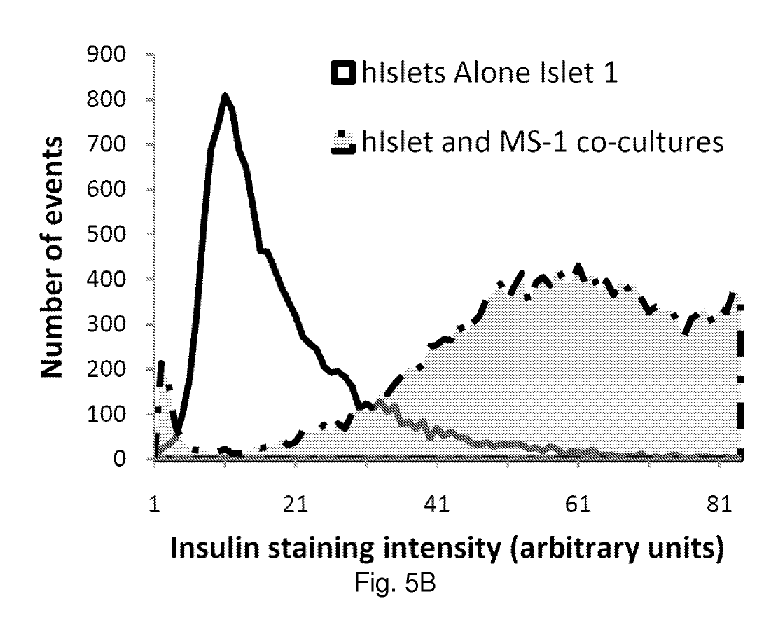

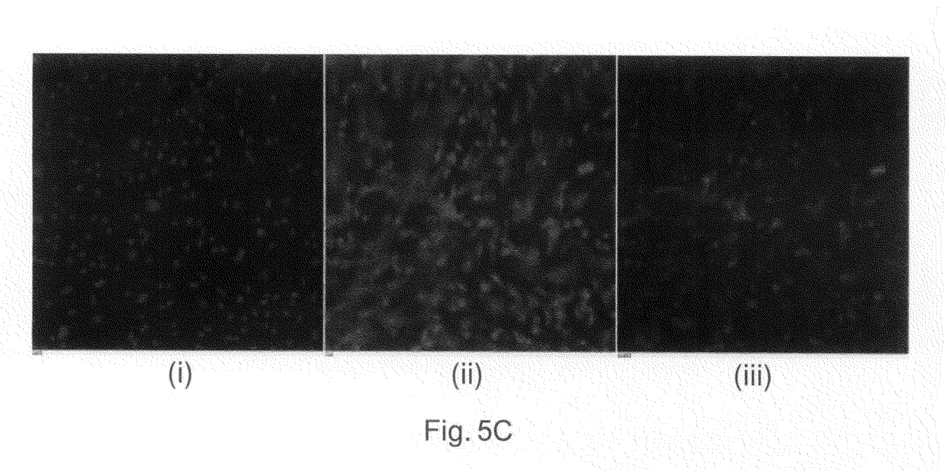

Production of primary islet derived human ECs (See FIGS. 5A, 5B, and 5C)

Previous reports show the ability of primary islet-derived ECs from rats to grow efficiently in vitro (17), expressly incorporated herein by reference. This previously reported protocol has been adapted for the enrichment and propagation of primary human islet derived-ECs. 100 islets are sufficient to produce a full 24 well plate by passage 3.

Purified human islets are cultured on collagen I coated plates for 2 wks. BS-1 positive ECs from mixed cultures are enriched using magnetic bead isolation (AutoMACS pro, Miltenyi). Passage 3 cells are tested for ECM components as well as other EC markers (i.e. PE-CAM/BS-1). Enriched human islet derived can be cryopreserved indefinitely.

### Co-Cultures of Primary Islet Derived Human ECs and Primary Human Islets

Primary islet derived ECs are grown to ˜100% confluency in T-12.5 tissues culture treated flask. Primary hIslets are added to the flask containing the MS-1 cells. The flask is placed on an orbital shaker in a 37° C. 5% CO₂ incubator running at 70 rpm. The media is replaced every 4 days.

FIGS. 5A, 5B, and 5C shows in vitro expanded primary ECs from human islets express collagen IV and induce the formation of islet like structures. Human primary islets were cultured on collagen coated plates. FIG. 5A shows a fresh free floating islet. FIG. 5B shows primary human islets were cultured on collagen coated plates. Note the migration of EC from the human islets by 1 wk. Following 2 wks islet derived ECs were magnetically sorted and plated at high purity. FIG. 5C shows IF staining of primary islet derived hECs. i-DAPI, ii-Collage IV, iii-BS-1.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes, which come within the meaning and range of equivalency of the claims, are to be embraced within their scope.

The disclosure herein is intended to encompass all combinations, subcombinations, and permutations of the various options, elements and steps disclosed herein, to the extent consistent, and is not to be deemed limited to particular combinations, or groups thereof, defined by the embodiments.

### Example 4

### Insulin Release Studies

Beta-cell monolayers were trypsinized after 5 d and subcultured at 37° C. for 48 h in a 96-well tissue culture plate at a cell density of 3×10⁴/well. PIs received fresh medium on the fifth day of co-culturing with continued exposure to the MS1s for an additional 2 d. The monolayers were subsequently deprived of glucose for 2 h followed by 1 h incubation in a balanced salt solution (11) with varying glucose amounts. Glucose deprivation of the PIs was carried out in a petri dish after which the PIs were distributed in a 96-well tissue culture plate and exposed to the same range of glucose concentrations as the monolayers. Secreted insulin was quantified from the cell supernatants using an immunoassay kit (Crystal Chem Inc., Downers Grove, Ill.) according to the manufacturer's instructions.

RNA isolation and semi-quantitative RT-PCR. RNA was purified from cell lysates using an RNeasy Mini kit (Qiagen, Valencia, Calif.). cDNA was synthesized with a Transcriptor High Fidelity cDNA Synthesis kit (Roche, Indianapolis, Ind.) and a MyCycler thermal cycler (Bio-Rad, Hercules, Calif.) The following primers were used for semi-quantitative PCR reactions:

Col-IV:
SEQ ID NO: 001
F-TGGGCGAGGGACATGCAATTACTA,
SEQ ID NO: 002
R-ACATCGGCTAATACGCGTCCTCAA;
Laminin A1:
SEQ ID NO: 003
F-GACCGCCATGCCGATTTAGC,
SEQ ID NO: 004
R-GACCGCCGTGTTGTTGATGC;
Laminin A5:
SEQ ID NO: 005
F-CCCTGGGGCCTTGAACTTCTCCTACTC,
SEQ ID NO: 006
R-GCATTGCGCCGATCCACCTCAG;
Laminin B1:
SEQ ID NO: 007
F-ACCAGACGGGCCTTGCTTGTGAAT,
SEQ ID NO: 008
R-AGTTGTGGCCCGTGGTGTAGTCCTG;
β-Actin:
SEQ ID NO: 009
F-GTGGGCCGCCCTAGGCACCA,
SEQ ID NO: 010
R-CTCTTTGATGTCACGCACGATTTC.

Statistical Analysis. Data are expressed as mean±SEM. The differences between means and the effects of treatments were analyzed by Student's t-test, one-way ANOVA with Tukey's post hoc test, or two-way ANOVA using Prism 5 (GraphPad software) where appropriate. Differences between treatments were considered significant at p<0.05.

### REFERENCES, THE ENTIRE DISCLOSURES OF WHICH ARE INCORPORATED BY REFERENCE HEREIN (36 AND 37)

1. Daoud, J., L. Rosenberg, and M. Tabrizian. 2010. Pancreatic islet culture and preservation strategies: advances, challenges, and future outlook. Cell Transplant 19:1523-1535.
2. Olsson, R., and P. O. Carlsson. 2006. The pancreatic islet endothelial cell: emerging roles in islet function and disease. Int J Biochem Cell Biol 38:710-714.
3. Johansson, A., J. Olerud, M. Johansson, and P. O. Carlsson. 2009. Angiostatic factors normally restrict islet endothelial cell proliferation and migration: implications for islet transplantation. Transpl Int 22:1182-1188.
4. Paraskevas, S., D. Maysinger, R. Wang, T. P. Duguid, and L. Rosenberg. 2000. Cell loss in isolated human islets occurs by apoptosis. Pancreas 20:270-276.
5. Nanji, S. A., and A. M. Shapiro. 2006. Advances in pancreatic islet transplantation in humans. Diabetes Obes Metab 8:15-25.
6. Rosenberg, L., R. Wang, S. Paraskevas, and D. Maysinger. 1999. Structural and functional changes resulting from islet isolation lead to islet cell death. Surgery 126:393-398.
7. Paraskevas, S., W. P. Duguid, D. Maysinger, L. Feldman, D. Agapitos, and L. Rosenberg. 1997. Apoptosis occurs in freshly isolated human islets under standard culture conditions. Transplant Proc 29:750-752.
8. Daoud, J., M. Petropavlovskaia, L. Rosenberg, and M. Tabrizian. 2010. The effect of extracellular matrix components on the preservation of human islet function in vitro. Biomaterials 31:1676-1682.
9. Lechner, A., A. L. Nolan, R. A. Blacken, and J. F. Habener. 2005. Redifferentiation of insulin-secreting cells after in vitro expansion of adult human pancreatic islet tissue. Biochem Biophys Res Commun 327:581-588.
10. Suschek, C., K. Fehsel, K. D. Kroncke, A. Sommer, and V. Kolb-Bachofen. 1994. Primary cultures of rat islet capillary endothelial cells. Constitutive and cytokine-inducible macrophagelike nitric oxide synthases are expressed and activities regulated by glucose concentration. Am J Pathol 145:685-695.
11. Hauge-Evans A C, Squires PE, Persaud S J, & Jones P M (1999) Pancreatic beta-cell-to-beta-cell interactions are required for integrated responses to nutrient stimuli: enhanced Ca2+ and insulin secretory responses of MIN6 pseudoislets. (Translated from eng) Diabetes 48(7):1402-1408 (in eng).
12. Luther M J, et al. (2006) MIN6 beta-cell-beta-cell interactions influence insulin secretory responses to nutrients and non-nutrients. (Translated from eng) Biochem Biophys Res Commun 343(1):99-104 (in eng).
13. Persaud S J, et al. (2010) Pseudoislets as primary islet replacements for research: report on a symposium at King's College London, London UK. (Translated from eng) Islets 2(4):236-239 (in eng).
14. Roderigo-Milne H, Hauge-Evans A C, Persaud S J, & Jones P M (2002) Differential expression of insulin genes 1 and 2 in MIN6 cells and pseudoislets. (Translated from eng) Biochem Biophys Res Commun 296(3):589-595 (in eng).
15. Lock L T, Laychock S G, & Tzanakakis E S (2011) Pseudoislets in stirred-suspension culture exhibit enhanced cell survival, propagation and insulin secretion. (Translated from eng) J Biotechnol 151(3):278-286 (in eng).
16. Kelly C, Guo H, McCluskey J T, Flatt P R, & McClenaghan N H (2010) Comparison of insulin release from MIN6 pseudoislets and pancreatic islets of Langerhans reveals importance of homotypic cell interactions. (Translated from eng) Pancreas 39(7):1016-1023 (in eng).
17. Guo-Parke H, et al. (2012) Configuration of electrofusion-derived human insulin-secreting cell line as pseudoislets enhances functionality and therapeutic utility. (Translated from eng) J Endocrinol 214(3):257-265 (in eng).
18. Squires P E, Hauge-Evans A C, Persaud S J, & Jones P M (2000) Synchronization of Ca(2+)-signals within insulin-secreting pseudoislets: effects of gap junctional uncouplers. (Translated from eng) Cell Calcium 27(5):287-296 (in eng).
19. Maillard E, et al. (2009) Extracellular matrix proteins involved in pseudoislets formation. (Translated from eng) Islets 1(3):232-241 (in eng).
20. Cavallari G, Zuellig R A, Lehmann R, Weber M, & Moritz W (2007) Rat pancreatic islet size standardization by the “hanging drop” technique. (Translated from eng) Transplant Proc 39(6):2018-2020 (in eng).
21. Johansson M, Mattsson G, Andersson A, Jansson L, & Carlsson P O (2006) Islet endothelial cells and pancreatic beta-cell proliferation: studies in vitro and during pregnancy in adult rats. (Translated from eng) Endocrinology 147(5):2315-2324 (in eng).
22. Jiang F X, Naselli G, & Harrison L C (2002) Distinct distribution of laminin and its integrin receptors in the pancreas. (Translated from eng) J Histochem Cytochem 50(12):1625-1632 (in eng).
23. Stendahl J C, Kaufman D B, & Stupp S I (2009) Extracellular matrix in pancreatic islets: relevance to scaffold design and transplantation. (Translated from eng) Cell Transplant 18(1):1-12 (in eng).
24. Olsson R & Carlsson P O (2006) The pancreatic islet endothelial cell: emerging roles in islet function and disease. (Translated from eng) Int J Biochem Cell Biol 38(5-6):710-714 (in eng).
25. Akirav E M, et al. (2011) Glucose and inflammation control islet vascular density and beta-cell function in NOD mice: control of islet vasculature and vascular endothelial growth factor by glucose. (Translated from eng) Diabetes 60(3):876-883 (in eng).
26. Luther M J, et al. (2005) Cell-to-cell contact influences proliferative marker expression and apoptosis in MIN6 cells grown in islet-like structures. (Translated from eng) Am J Physiol Endocrinol Metab 288(3):E502-509 (in eng).
27. Ogata T, Park K Y, Seno M, & Kojima I (2004) Reversal of streptozotocin-induced hyperglycemia by transplantation of pseudoislets consisting of beta cells derived from ductal cells. (Translated from eng) Endocr J 51(3):381-386 (in eng).
28. Paraskevas S, et al. (1997) Apoptosis occurs in freshly isolated human islets under standard culture conditions. (Translated from eng) Transplant Proc 29(1-2):750-752 (in eng).
29. Nikolova G & Lammert E (2003) Interdependent development of blood vessels and organs. (Translated from eng) Cell Tissue Res 314(1):33-42 (in eng).
30. Daoud J, Rosenberg L, & Tabrizian M (2010) Pancreatic islet culture and preservation strategies: advances, challenges, and future outlook. (Translated from eng) Cell Transplant 19(12):1523-1535 (in eng).
31. Kitsou-Mylona I, Burns C J, Squires P E, Persaud S J, & Jones P M (2008) A role for the extracellular calcium-sensing receptor in cell-cell communication in pancreatic islets of langerhans. (Translated from eng) Cell Physiol Biochem 22(5-6):557-566 (in eng).
32. Rogers G J, Hodgkin M N, & Squires P E (2007) E-cadherin and cell adhesion: a role in architecture and function in the pancreatic islet. (Translated from eng) Cell Physiol Biochem 20(6):987-994 (in eng).
33. Reers C, et al. (2011) Down-regulation of proliferation does not affect the secretory function of transformed beta-cell lines regardless of their anatomical configuration. (Translated from eng) Islets 3(3):80-88 (in eng).
34. Arbiser J L, et al. (1997) Oncogenic H-ras stimulates tumor angiogenesis by two distinct pathways. (Translated from eng) Proc Natl Acad Sci USA 94(3):861-866 (in eng).
35. Tal M, et al. (1992) Glucose transporter isotypes switch in T-antigen-transformed pancreatic beta cells growing in culture and in mice. (Translated from eng) Mol Cell Biol 12(1):422-432 (in eng).
36. Spelios M G, et al. (2015) Islet endothelial cells induce glycosylation and increase cell-surface expression integrin β1 in β-cells. Journal of Biological Chemistry. 2015 Apr. 24. pii: jbc.M114.628784. [Epub ahead of print]
37. Spelios M G, et al. (2013) In vitro formation of β cell pseudoislets using islet-derived endothelial cells. PLoS One. 2013 Aug. 28; 8(8):e72260.

What is claimed is:

1. A method substantially as shown and described herein.

2. A novel method for forming pseudoislets, comprising co-culturing pancreatic beta cells with islet endothelial cells, for a sufficient time until at least spontaneous formation of free-floating pseudoislets, wherein the islet endothelial cells produce extracellular matrix proteins, and at least a portion of the pancreatic beta cells in the pseudoislets have increased insulin gene expression with respect to the pancreatic beta cells cultures on a surface absent the islet endothelial cells.

3. Novel insulin-secreting beta cells line according to claim 1 and claim 2 which exist in the form of aggregates called “pseudoislets”.

4. Method for co-culturing beta cell line, including betaTC3 together with islet-derived endothelial cells according to claims 1 to 3 to form free-floating pseudoislets with improved beta cell function and extracellular proteins, including but not limited to collagen IV, laminin, fibronectin.

5. Method for co-culturing beta cell line, including MING cells together with islet-derived endothelial cells according to claims 1 to 3 to form free-floating pseudoislets with improved beta cell function and extracellular proteins, including but not limited to collagen IV, laminin, fibronectin.

6. Method for co-culturing human beta cell line, together with mouse or human islet-derived endothelial cells according to claims 1 to 3 to form free-floating pseudoislets with improved beta cell function and extracellular proteins, including but not limited to collagen IV, laminin, fibronectin.

7. Method for generating pseudoislets according to claims 1 to 7 which are free floating in vitro and can be used for islet transplantation as a therapy for islet dysfunction

8. Method for newly formed pseudoislets according to claims 1 to 6 which may be used in research and therapies to produce artificial organs which may be extracorporeal or implanted in patients.

9. Method for newly formed pseudoislets according to claims 1 to 6 which may be used for high-throughput screening of novel drugs and compounds designed to improve hormone production in beta cells

10. Method for newly formed pseudoislets according to claims 1 to 6 which may include other islet cells including alphaTC3 glucagon positive cells, somatostatin positive cells which may be used to study cell-cell interactions in vitro

11. Method for maintaining and improving cell function and insulin production by co-culturing of islet derived murine endothelial cells and primary human islets.

12. Method for maintaining beta-cell function in primary human islets according by the addition of recently isolated primary human islet to islet-derived murine endothelial cells

13. Method for maintaining beta-cell function in primary human islets according to claim 11 by co-culturing of murine islet derived endothelial cells in a shaker to prevent islet adhesion and maintain free floating islets.

14. Method for maintaining beta-cell function in primary human islets according to claim 11 by the addition of recently isolated primary human islet to islet-derived human endothelial cells

15. Method for maintaining beta-cell function in primary human islets according to claim 11 by co-culturing of human islet derived endothelial cells in a shaker to prevent islet adhesion and maintain free floating islets.

16. Method for maintaining and recovering primary human islet function by co-culturing of human islets with human islet-derived endothelial cells according to claim 11 that may be extracorporeal or implanted in patients.

17. Method for maintaining and recovering primary human islet function by co-culturing of human islets with human islet-derived endothelial cells according to claim 11 to which may be used for high-throughput screening of novel drugs and compounds designed to improve hormone production in beta cells.


SEQUENCE LISTING (20 sequences)

SEQ ID NO: 1 - DNA, 24 bp, Artificial Sequence
  Col-IV Forward PCR Primer
tgggcgagggacatgcaattacta

SEQ ID NO: 2 - DNA, 24 bp, Artificial Sequence
  Col-IV Reverse PCR Primer
acatcggctaatacgcgtcctcaa

SEQ ID NO: 3 - DNA, 20 bp, Artificial Sequence
  Laminin A1 Forward PCR Primer
gaccgccatgccgatttagc

SEQ ID NO: 4 - DNA, 20 bp, Artificial Sequence
  Laminin A1 Reverse PCR Primer
gaccgccgtgttgttgatgc

SEQ ID NO: 5 - DNA, 27 bp, Artificial Sequence
  Laminin A5 Forward PCR Primer
ccctggggccttgaacttctcctactc

SEQ ID NO: 6 - DNA, 22 bp, Artificial Sequence
  Laminin A5 Reverse PCR Primer
gcattgcgccgatccacctcag

SEQ ID NO: 7 - DNA, 24 bp, Artificial Sequence
  Laminin B1 Forward PCR Primer
accagacgggccttgcttgtgaat

SEQ ID NO: 8 - DNA, 25 bp, Artificial Sequence
  Laminin B1 Reverse PCR Primer
agttgtggcccgtggtgtagtcctg

SEQ ID NO: 9 - DNA, 20 bp, Artificial Sequence
  Beta-Actin Forward PCR Primer
gtgggccgccctaggcacca

SEQ ID NO: 10 - DNA, 24 bp, Artificial Sequence
  Beta-Actin Reverse PCR Primer
ctctttgatgtcacgcacgatttc

SEQ ID NO: 11 - DNA, 24 bp, Artificial Sequence
  Insulin 1 Forward PCR Primer
acctggagaccttaatgggccaaa

SEQ ID NO: 12 - DNA, 24 bp, Artificial Sequence
  Insulin 1 Reverse PCR Primer
atgacctgcttgctgatggtctct

SEQ ID NO: 13 - DNA, 24 bp, Artificial Sequence
  Bcl-2 Forward PCR Primer
tttgatttctcctggctgtctctg

SEQ ID NO: 14 - DNA, 24 bp, Artificial Sequence
  Bcl-2 Reverse PCR Primer
atatttgtttggggcaggtttgtc

SEQ ID NO: 15 - DNA, 24 bp, Artificial Sequence
  Bcl-x1 Forward PCR Primer
gttctcttggcctcaaaactcaca

SEQ ID NO: 16 - DNA, 24 bp, Artificial Sequence
  Bcl-x1 Reverse PCR Primer
acacatcaaaaccaaggcaagcta

SEQ ID NO: 17 - DNA, 22 bp, Artificial Sequence
  Ki67 Rorward PCR Primer
gcaaaccctcacacttggagaa

SEQ ID NO: 18 - DNA, 25 bp, Artificial Sequence
  Ki67 Reverse PCR Primer
tgccacgagtaacatatattgctgg

SEQ ID NO: 19 - DNA, 20 bp, Artificial Sequence
  Beta-Actin Forward PCR Primer
gcaagtgcttctaggcggac

SEQ ID NO: 20 - DNA, 22 bp, Artificial Sequence
  Beta-Actin Reverse PCR Primer
aagaaagggtgtaaaacgcagc